(19) United States
(12) Patent Application Publication
(10) Pub. No.: US 2016/0145221 A1
(43) Pub. Date: May 26, 2016

(54) METHOD FOR PRODUCING POLYISOCYANATES COMPRISING IMINOOXADIAZINEDIONE GROUPS AND USE OF THESE

(71) Applicant: COVESTRO DEUTSCHLAND AG, Leverkusen (DE)

(72) Inventors: Frank Richter, Leverkusen (DE); Reinhard Halpaap, Odenthal (DE)

(21) Appl. No.: 14/905,547

(22) PCT Filed: Jul. 21, 2014
(86) PCT No.: PCT/EP2014/065578

§ 371 Date: Jan. 15, 2016

(30) Foreign Application Priority Data
Jul. 25, 2013 (DE) 13177981.1

(51) Int. Cl. C07D 273/04

(52) U.S. Cl. CPC C07D 273/04

(57) ABSTRACT

The present invention relates to a method for producing polyisocyanates comprising iminooxadiazinedione groups, wherein at least one monomeric di- and/or tri-isocyanate is oligomerised in the presence of a) at least one catalyst, b) at least one additive (A) having a relative permittivity at 18° C. to 30° C. of less than 4.0, c) optionally further additives other than A. The invention relates further to a reaction system for producing polyisocyanates comprising iminooxadiazinedione groups, and to the use of an additive (A) having a relative permittivity at 18° C. to 30° C. of less than 4.0 for producing polyisocyanates comprising iminooxadiazinedione groups by catalysed modification of monomeric di- and/or tri-isocyanates.

### CROSS-REFERENCE TO RELATED APPLICATIONS

This application is a National Phase Application of PCT/EP2014/065578, filed Jul. 21, 2014, which claims priority to European Application No. 13177981.1, filed Jul. 25, 2013, each of which being incorporated herein by reference.

### FIELD OF THE INVENTION

The present invention relates to a method for producing polyisocyanates containing iminooxadiazinedione groups, wherein at least one monomeric di- and/or tri-isocyanate is oligomerized in the presence of
a) at least one catalyst,
b) at least one additive (A) having a relative permittivity at 18° C. to 30° C. of less than 4.0,
c) optionally further additives other than A.

The invention relates further to a reaction system for producing polyisocyanates comprising iminooxadiazinedione groups, and to the use of an additive (A) having a relative permittivity at 18° C. to 30° C. of less than 4.0 for producing polyisocyanates comprising iminooxadiazinedione groups by catalyzed modification of monomeric di- and/or tri-isocyanates.

### BACKGROUND OF THE INVENTION

The oligo- or poly-merization of isocyanates, here referred to collectively as modification, has been known for a long time. If the modified polyisocyanates comprise free NCO groups, which may also have been temporarily deactivated with blocking agents, they are extraordinarily high-quality starting materials for the production of a large number of polyurethane plastics materials and coating compositions.

A number of industrial processes for modifying isocyanates have become established, wherein the isocyanate to be modified, which in most cases is a diisocyanate, is generally converted by addition of catalysts and then, when the desired degree of conversion of the isocyanate to be modified has been reached, the catalysts are rendered inactive (deactivated or separated off) by suitable measures and the resulting polyisocyanate is generally separated from the unconverted monomer. A summary of these processes of the art is to be found in H. J. Laas et al., J. Prakt. Chem. 1994, 336, 185 ff.

A special form of isocyanate modification, which yields products having a high content of iminooxadiazinedione groups (asymmetrical isocyanate trimers) in the products, in addition to the isocyanurate structures (symmetrical isocyanate trimers, frequently referred to hitherto only as “trimers” for the sake of simplicity) which have been known for a long time, is described inter alia in EP 962 455 A1, EP 962 454 A1, EP 896 009 A1, EP 798 299 A1, EP 447 074 A1, EP 379 914 A1, EP 339 396 A1, EP 315 692 A1, EP 295 926 A1 and EP 235 388 A1. (Hydrogen poly)fluorides inter alia have been found to be suitable catalysts therefor.

A disadvantage of the known processes of the art is that the iminooxadiazinedione content in the products is only about 50%, based on the sum of symmetrical (isocyanurate) and asymmetrical trimer (iminooxadiazinedione), and that content decreases further in the case of higher monomer conversion.

Although the iminooxadiazinedione content in the products can be influenced in the desired direction by increasing the “HF content” in the catalyst, that is to say by changing from simple fluorides (which generally do not have long-term stability and gradually change to the difluoride form even without the external addition of HF) to difluorides, trifluorides, etc., this procedure has disadvantages (higher HF content in the waste process gas, which must be neutralized in a complex procedure, higher corrosivity of the catalyst solutions, etc.) which do not outweigh the advantages.

### SUMMARY OF THE INVENTION

Accordingly, the present invention provides a method for producing polyisocyanates having a high iminooxadiazinedione group content that is not accompanied by the above-mentioned disadvantages: the products are to exhibit a higher content of iminooxadiazinedione structures than the products that are available by known processes of the art, without increasing the “HF content” in the catalyst.

It is understood that the invention disclosed and described in this specification is not limited to the embodiments summarized in this Summary.

These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages, and so forth in the specification are to be understood as being modified in all instances by the term “about.”

Any numerical range recited in this specification is intended to include all sub-ranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this specification is intended to include all lower numerical limitations subsumed therein and any minimum numerical limitation recited in this specification is intended to include all higher numerical limitations subsumed therein. Accordingly, Applicants reserve the right to amend this specification, including the claims, to expressly recite any sub-range subsumed within the ranges expressly recited herein. All such ranges are intended to be inherently described in this specification such that amending to expressly recite any such sub-ranges would comply with the requirements of 35 U.S.C. §112(a), and 35 U.S.C. §132(a).

Any patent, publication, or other disclosure material identified herein is incorporated by reference into this specification in its entirety unless otherwise indicated, but only to the extent that the incorporated material does not conflict with existing definitions, statements, or other disclosure material expressly set forth in this specification. As such, and to the extent necessary, the express disclosure as set forth in this specification supersedes any conflicting material incorporated by reference herein. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

Reference throughout this specification to “various non-limiting embodiments,” “certain embodiments,” or the like, means that a particular feature or characteristic may be included in an embodiment. Thus, use of the phrase “in various non-limiting embodiments,” “in certain embodiments,” or the like, in this specification does not necessarily refer to a common embodiment, and may refer to different embodiments. Further, the particular features or characteristics may be combined in any suitable manner in one or more embodiments. Thus, the particular features or characteristics illustrated or described in connection with various or certain embodiments may be combined, in whole or in part, with the features or characteristics of one or more other embodiments without limitation. Such modifications and variations are intended to be included within the scope of the present specification.

The grammatical articles “a”, “an”, and “the”, as used herein, are intended to include “at least one” or “one or more”, unless otherwise indicated, even if “at least one” or “one or more” is expressly used in certain instances. Thus, these articles are used in this specification to refer to one or more than one (i.e., to “at least one”) of the grammatical objects of the article. By way of example, and without limitation, “a component” means one or more components, and thus, possibly, more than one component is contemplated and may be employed or used in an implementation of the described embodiments. Further, the use of a singular noun includes the plural, and the use of a plural noun includes the singular, unless the context of the usage requires otherwise.

The present invention provides a method for producing polyisocyanates comprising iminooxadiazinedione groups, wherein at least one monomeric di- and/or tri-isocyanate is oligomerized in the presence of
a) at least one catalyst,
b) at least one additive (A) having a relative permittivity at 18° C. to 30° C. of less than 4.0,
c) optionally further additives other than A.

The relative permittivity at 18° C. to 30° C. is determined within the meaning of the present invention by the ratio of the capacitances of a capacitor with on the one hand the substance and on the other hand vacuum as the dielectric, at a measuring frequency of 50 Hz. This can be expressed by the following relationship, which is generally known:

$${ɛ}_{r} = \frac{ɛ}{{ɛ}_{0}}$$

where ∈ᵣ is the relative permittivity, E is the measured permittivity of the substance and Co is the vacuum permittivity. The mentioned temperature range of 18° C. to 30° C. means that the substance has the indicated relative permittivity at any desired temperature in that range. For example, if an additive has a relative permittivity at 30° C. of 3.0 but a higher relative permittivity than, for example, 4.0 at 20° C., the additive nevertheless complies with the definition according to the invention of a relative permittivity at 18° C. to 30° C. of less than 4.0. The same is true, by analogy, for the preferred ranges of the relative permittivity defined hereinbelow.

The following table gives an overview of typical values of the relative permittivities of different substances, in each case measured at 50 Hz:

| | relative | |
|---|---|---|
| Solvent | permittivity at | ° C. |
| n-Hexane | 1.9 | 25 |
| n-Heptane | 2.0 | 25 |
| Cyclohexane | 2.0 | 20 |
| Isoeicosane | 2.1 | 25 |
| 1,4-Dioxane | 2.2 | 25 |
| Carbon tetrachloride | 2.2 | 20 |
| Benzene | 2.3 | 25 |
| Tetrachloroethene | 2.3 | 25 |
| Toluene | 2.4 | 25 |
| Triethylamine | 2.4 | 25 |
| Carbon disulfide | 2.6 | 20 |
| Trichloroethene | 3.4 | 16 |
| Anisole | 4.3 | 25 |
| Dibutyl ether | 4.3 | 20 |
| Diethyl ether | 4.3 | 20 |
| Chloroform | 4.8 | 20 |
| Bromobenzene | 5.4 | 25 |
| Chlorobenzene | 5.6 | 25 |
| Piperidine | 5.8 | 20 |
| Ethyl acetate | 6.0 | 25 |
| Glacial acetic acid | 6.2 | 20 |
| Aniline | 6.9 | 20 |
| Ethylene glycol dimethyl ether | 7.2 | 25 |
| Triethylene glycol dimethyl ether (triglyme) | 7.5 | 25 |
| 1,1,1-Trichloroethane | 7.5 | 20 |
| Tetrahydrofuran | 7.6 | 25 |
| Diethylene glycol | 7.7 | 25 |
| Methylene chloride | 8.9 | 25 |
| Quinoline | 9.0 | 25 |
| Ethylene dichloride | 10.4 | 25 |
| Pyridine | 12.4 | 21 |
| 2-Methyl-2-propanol (tert-butanol) | 12.5 | 25 |
| 3-Methyl-1-butanol (isoamyl alcohol) | 14.7 | 25 |
| 1-Butanol | 17.5 | 25 |
| Methyl ethyl ketone (butanone) | 18.5 | 20 |
| 2-Propanol (isopropyl alcohol) | 19.9 | 25 |
| Propanol | 20.3 | 25 |
| Acetic anhydride | 20.7 | 19 |
| Acetone | 20.7 | 25 |
| Triethylene glycol | 23.7 | 20 |
| Ethanol | 24.6 | 25 |
| Benzonitrile | 25.2 | 25 |
| Adiponitrile | 30.0 | 18 |
| N-Methyl-2-pyrrolidone (NMP) | 32.2 | 25 |
| Methanol | 32.7 | 25 |
| Nitrobenzene | 34.8 | 25 |
| Nitromethane | 35.9 | 30 |
| Gamma-valerolactone | 36.9 | 20 |
| Dimethylformamide | 37.0 | 25 |
| Acetonitrile | 37.5 | 20 |
| Ethylene glycol | 37.7 | 25 |
| Dimethylacetamide | 37.8 | 25 |
| γ-Butyrolactone | 39.1 | 25 |
| Sulfolane | 43.3 | 30 |
| Dimethyl sulfoxide | 46.7 | 25 |
| Propylene carbonate (4-methyl-1,3-dioxol-2- | 65.1 | 25 |
| one) | | |
| Water | 78.4 | 25 |
| Formamide | 111.0 | 20 |
| N-Methylformamide | 182.4 | 25 |

Within the scope of the method according to the invention there can be used as the additive (A) a linear or branched, aliphatic, cycloaliphatic and/or araliphatic C₄-C₃₀-alkane, in particular C₅-C₃₀-alkane, or mixtures thereof. Accordingly, the additive (A) is selected from the group consisting of linear, branched or cyclic butane, pentane, hexane, heptane, octane, nonane, decane, undecane, dodecane, tridecane, tetradecane, pentadecane, hexadecane, heptadecane, octadecane, nonadecane, eicosane, heneicosane, docosane, tricosane, tetracosane, pentacosane, hexacosane, heptacosane, octacosane, nonacosane, tricontane, and mixtures thereof.

It cannot be inferred from any of the documents of the art mentioned at the beginning that the catalysts of the art that are preferred for iminooxadiazinedione formation effect a significant increase in the iminooxadiazinedione content in the products in the presence of the above-mentioned additives, in particular when the monomer conversion is increased. In an advantageous embodiment of the method according to the invention, the additive (A) has a relative permittivity at 18° C. to 30° C. of not more than 3.5, preferably of not more than 3.0, particularly preferably of not more than 2.8 or even not more than 2.5.

The amount of additive to be used can vary within wide limits in the method according to the invention. It is preferably in some embodiments from 1 to 50 wt. %, based on the mass of the monomeric di- and/or tri-isocyanate to be modified, in other embodiments from 2 to 30 wt. %, and in still other embodiments from 2 to 20 wt. %. As low an amount of additive as possible is of course technically advantageous in order on the one hand to make the space-time yield of polyisocyanate resin high and to keep the catalyst requirement low. However, even with the addition of 20% isoeicosane, the additional amount of catalyst required is still wholly within the technically acceptable range, while the iminooxadiazinedione content in the resulting polyisocyanate resins is significantly increased and especially does not fall as greatly as without additive, even in the case of a higher monomer conversion.

By means of the modification method according to the invention, an improved method for producing polyisocyanates having a high content of iminooxadiazinedione groups has therefore been made available in a simple manner.

In one embodiment of the method according to the invention, the additive (A) is mixed with the monomer(s) to be modified. In a further embodiment of the method according to the invention, the additive (A) is added to the monomeric di- and/or tri-isocyanate before it is brought into contact with the catalyst.

Any known isocyanates can in principle be used within the scope of the method according to the invention. Di- and/or tri-isocyanates having aliphatically, cycloaliphatically, araliphatically and/or aromatically bonded isocyanate groups are preferably used, individually or in arbitrary mixtures with one another. The methods by which the above-mentioned (poly)isocyanates are generated, that is to say with or without the use of phosgene, are unimportant. Particular mention may be made of: hexamethylene diisocyanate (HDI), 2-methylpentane 1,5-diisocyanate, 2,4,4-trimethyl-1,6-hexane diisocyanate, 2,2,4-trimethyl-1,6-hexane diisocyanate, 4-isocyanatomethyl-1,8-octane diisocyanate, 3(4)-isocyanatomethyl-1-methylcyclohexyl isocyanate (IMCI), isophorone diisocyanate (IPDI), 1,3- and 1,4-bis(isocyanatomethyl)benzene (XDI), 1,3- and 1,4-bis(isocyanatomethyl)cyclohexane (H6XDI), 2,4- and 2,6-toluylene diisocyanate (TDI), bis(4-isocyanatophenyl)methane (4,4′MDI), 4-isocyanatophenyl-2-isocyanato-phenylmethane (2,4′MDI) and also polynuclear products which are obtainable by formaldehyde-aniline polycondensation and subsequent conversion of the resulting (poly)amines into the corresponding (poly)isocyanates (polymeric MDI). Aliphatic di- and/or tri-isocyanates are preferably used, particularly preferably aliphatic diisocyanates.

Most particular preference is given to the use of hexamethylene diisocyanate (HDI), 2-methylpentane 1,5-diisocyanate, 2,4,4-trimethyl-1,6-hexane diisocyanate, 2,2,4-trimethyl-1,6-hexane diisocyanate and/or 4-isocyanatomethyl-1,8-octane diisocyanate, yet further preference being given to HDI.

Suitable catalysts are in principle any compounds of the art which have previously been described for this purpose, as such or in solution. Particularly suitable are substances having a salt-like structure with cations which ensure good solubility in the isocyanate medium, in particular tetraorganyl-ammonium salts and -phosphonium salts, with anions selected from the group RfCR₁R₂COOH, wherein Rf represents a straight-chained or branched perfluoroalkyl radical and R₁ and R₂ independently of one another represent H or straight-chained or branched organyl radicals, fluoride (F⁻), di- and/or poly-(hydrogen) fluorides ([F⁻×HF)ₘ]), wherein m has a numerical value in some embodiments of from 0.001 to 20, in other embodiments of from 0.1 to 20, in still other embodiments of from 0.5 to 20, and in yet other embodiments of from 0.5 to 5.

The di- and/or poly-(hydrogen) fluoride ([F⁻×HF)ₘ]) can in particular be a quaternary ammonium fluoride, ammonium difluoride, ammonium trifluoride, a higher ammonium polyfluoride, a phosphonium fluoride, a phosphonium difluoride, a phosphonium trifluoride and/or a higher phosphonium polyfluoride, preferably those which can be prepared by mixing quaternary ammonium and phosphonium fluorides or hydroxides with corresponding amounts of hydrogen fluoride, optionally pre-dissolved in alcohols or water.

Suitable solvents for the catalyst(s) are any compounds which do not react with the catalyst and are capable of dissolving it to a sufficient degree. For the above-mentioned tetraorganyl-ammonium salts and phosponium salts, for example, they are aliphatic or aromatic hydrocarbons, alcohols, esters and ethers. Alcohols are preferably used.

The amount of catalyst required in the method according to the invention does not differ significantly from that observed in the bulk modification of the art. The catalyst can be used, for example, in an amount of from 1 mol-ppm to 1 mol-%, preferably from 5 mol-ppm to 0.1 mol-%, based on the amount of monomer.

The method according to the invention can be carried out in some embodiments, for example, in the temperature range of from 0° C. to +250° C., in other embodiments from 20 to 180° C., and in still other embodiments from 40 to 150° C.

In a further form of the method according to the invention, the oligomerization can be terminated when from 5 to 80 wt. %, preferably from 10 to 60 wt. %, of the monomeric di- and/or tri-isocyanate used have been converted. The oligomerization can be terminated, for example, by deactivating the catalyst. A large number of -described methods of the art are suitable in principle for deactivating the catalyst, such as, for example, the addition of (sub- or super-)stoichiometric amounts of acids or acid derivatives (e.g. benzoyl chloride, acid esters of acids containing phosphorus or sulfur, those acids themselves, etc., but not HF), adsorptive binding of the catalyst and subsequent separation by filtration, or combinations thereof.

After deactivation of the catalyst, the unconverted monomer and any solvent used concomitantly can be separated off by any known separation techniques such as, for example, distillation, optionally in the special form of thin-film distillation, extraction or crystallization/filtration. Combinations of two or more of these techniques can of course also be used.

If the polyisocyanate produced according to the invention is to comprise free, unconverted monomer, as is of interest, for example, for further processing to NCO-blocked products, separation of the monomer after deactivation of the catalyst can be omitted.

The unconverted monomer is separated off in various embodiments, particularly by distillation. The products according to the invention preferably have a residual monomer content, after separation, of <0.5 wt. %, preferably <0.25 wt. %, particularly preferably <0.1 wt. %.

Compared with catalysis by means of, for example, quaternary phosphonium salts without the use of additives (bulk modification, see comparative example 1), a significant increase in the iminooxadiazinedione content in the products is observed in the method according to the invention, in particular in the case of a higher monomer conversion, under otherwise identical reaction conditions.

According to a further, continuous embodiment of the method according to the invention, the oligomerization can be carried out in a tubular reactor. This is advantageous because the catalysts according to the invention hereby have a significantly lower tendency spontaneously to form gel particles in the product as compared with the known catalysts of the art, even when used in a highly concentrated solution or in the form of the pure active substance.

The present invention relates further to a reaction system for producing polyisocyanates containing iminooxadiazinedione groups, which reaction system comprises at least one monomeric di- and/or tri-isocyanate as well as
a) at least one catalyst,
b) at least one additive (A) having a relative permittivity at 18° C. to 30° C. of less than 4.0, in particular of not more than 3.5, preferably not more than 3.0, particularly preferably of not more than 2.8 or even not more than 2.5,
c) optionally further additives other than (A).

The present invention further provides the use of compounds having a relative permittivity at 18° C. to 30° C. of less than 4.0, in particular of not more than 3.5, preferably of not more than 3.0, particularly preferably of not more than 2.8 or even not more than 2.5, as an additive (A) for producing polyisocyanates comprising iminooxadiazinedione groups by catalyzed modification of monomeric di- and/or tri-isocyanates.

The products or product mixtures obtainable by the method according to the invention are therefore starting materials which can be used in a versatile manner for producing optionally foamed plastics material(s) as well as coatings, coating compositions, adhesives and aggregates. They are suitable, optionally in NCO-blocked form, in particular for producing optionally water-dispersible one- and two-component polyurethane coatings, on account of their reduced solution and melt viscosity, as compared with products based (predominantly) on isocyanurate polyisocyanate, with an otherwise equally high or improved property profile. The HDI-based products according to the invention, even when highly diluted in coating solvents, are thus more stable to the occurrence of flocculation or turbidity than corresponding isocyanurate-based products.

They can be used in pure form or in conjunction with other isocyanate derivatives of the art, such as, for example, polyisocyanates comprising uretdione, biuret, allophanate, isocyanurate and/or urethane groups, the free NCO groups of which have optionally been deactivated with blocking agents.

### EXAMPLES

The present invention is explained in greater detail below by means of examples.

All amounts are by mass, unless indicated otherwise.

The NCO content of the resins described in the examples and comparative examples was determined by titration according to DIN 53 185.

The phosphorus content of all the samples was determined by X-ray fluorescence analysis (XRF).

Mol-% data were determined by NMR spectroscopy and, unless indicated otherwise, always relate to the sum of the NCO secondary products. The measurements were carried out using DPX 400 or DRX 700 instruments from Brucker on approximately 5% (¹H-NMR) or approximately 50% (¹³C-NMR) samples in dry C₆D₆ at a frequency of 400 or 700 MHz (¹H-NMR) or 100 or 176 MHz (¹³C-NMR). As reference for the ppm scale there were used small amounts of tetramethylsilane in the solvent with 0 ppm ¹H-NMR chem. shift. Alternatively, the signal of the C₆D₅H contained in the solvent was used as reference: 7.15 ppm ¹H-NMR chem. shift, 128.02 ppm ¹³C-NMR chem. shift. Data for the chemical shift of the compounds in question were taken from the literature (see D. Wendisch, H. Reiff and D. Dieterich, Die Angewandte Makromolekulare Chemie 141, 1986, 173-183 and literature cited therein, as well as EP-A 896 009).

The residual monomer contents were determined by gas chromatography.

Unless indicated otherwise, all the reactions were carried out under a nitrogen atmosphere.

The diisocyanates used are products of Covestro Deutschland AG; all other commercially available chemicals were obtained from Aldrich. The preparation of the hydrogen polyfluoride catalysts is known in the literature and is described inter alia in EP 962 454. The isoeicosane used is an isomeric mixture of different C₂₀-alkanes and was obtained from Ineos.

### Example 1

### Comparative Example

1000 g of HDI were placed in a double-walled flat ground flange vessel adjusted to 60° C. by an external circuit and having a stirrer, a reflux condenser connected to an inert gas system (nitrogen/vacuum) and a thermometer, and freed of dissolved gases by stirring for one hour in vacuo (0.1 mbar). After aeration with nitrogen, the refractive index at the frequency of the light of the D-line of the Na emission spectrum was measured at 20° C. (n_{D}²⁰ hereinbelow), and then the amount of catalyst indicated in Table 1 (based on the mass of HDI used, in the form of a 70% solution in isopropanol) was metered in portions in such a manner that the internal temperature did not exceed 65° C. When about 1 mol. of NCO groups had been converted, the catalyst was deactivated by addition of an amount of p-toluenesulfonic acid (in the form of a 40% solution in isopropanol) equivalent to the catalyst, and the mixture was then stirred for a further 30 minutes at reaction temperature and subsequently worked up.

Working up of the crude solution, the n_{D}²⁰ of which was 1.4610, was carried out by vacuum distillation in a thin-film evaporator, short path evaporator (SPE) type, with an upstream pre-evaporator (PE) (distillation conditions: pressure: 0.08+/−0.04 mbar, PE temperature: 120° C., SPE temp.: 140° C.), unconverted monomer was separated off as the distillate and the low-monomer polyisocyanate resin were separated off as the bottom product (initial pass: Example 1-A). The polyisocyanate resin was separated off and the distillate was collected in a second flat ground flange stirring apparatus, of identical construction to the first, and made up to the starting amount (1000 g) with freshly degassed HDI. The procedure was then as described at the beginning, with the difference that the isocyanate conversion (indicated by the refractive index of the raw materials) was raised stepwise from pass to pass. This procedure was repeated several times. The results are found in Table 1.

TABLE 1

| | | | | | Resin | | | |
|---|---|---|---|---|---|---|---|---|
| | Bu₄P⁺ [HF₂]⁻ | | | Amount | NCO | Imino- | | |
| | solution.^{(a)} | | Delta- | of resin | content | oxadia- | Iso- | Uret- |
| Ex. 1- | [mg] | n_{D}²⁰ ^{(b)} | n_{D}²⁰ ^{(c)} | [g] | [%] | zinediones^{(d)} | cyanurates^{(d)} | diones^{(d)} |
| A | 507 | 1.4610 | 0.0087 | 195 | 23.6 | 44.3% | 50.5% | 5.0% |
| B | 340 | 1.4604 | 0.0081 | 198 | 23.5 | 49.4% | 47.1% | 3.3% |
| C | 402 | 1.4647 | 0.0124 | 251 | 23.7 | 43.0% | 51.0% | 5.7% |
| D | 379 | 1.4650 | 0.0127 | 261 | 23.1 | 44.6% | 51.4% | 3.8% |
| E | 402 | 1.4665 | 0.0142 | 290 | 22.9 | 42.9% | 51.8% | 5.0% |
| F | 424 | 1.4668 | 0.0145 | 296 | 23.1 | 43.0% | 52.6% | 4.0% |
| G | 469 | 1.4678 | 0.0155 | 310 | 23.0 | 40.0% | 53.2% | 4.8% |
| H | 491 | 1.4680 | 0.0157 | 317 | 22.9 | 42.5% | 50.1% | 4.7% |
| I | 580 | 1.4740 | 0.0217 | 434 | 22.3 | 39.5% | 55.3% | 4.7% |
| J | 670 | 1.4745 | 0.0222 | 443 | 21.4 | 38.9% | 55.6% | 4.1% |
| K | 804 | 1.4805 | 0.0282 | 535 | 21.0 | 37.9% | 57.9% | 4.1% |
| L | 1216 | 1.4852 | 0.0329 | 608 | 20.5 | 36.2% | 57.6% | 3.9% |
| M | 1183 | 1.4920 | 0.0397 | 696 | 19.2 | 31.5% | 63.8% | 2.8% |

^{(a)}70% in iPrOH;
^{(b)} Refractive index of the reaction mixture after action of the stopper before distillation,
^{(c)} Increase in the refractive index as compared with the starting value before the first catalyst addition,
^{(d)}mol-% acc. to NMR, based on the sum of the NCO secondary products formed in the modification reaction, difference to 100%: urethanes/allophanates.

### Example 2

### According to the Invention

Additive: Isoeicosane (relative permittivity at 25° C./50 Hz: 2.1)

The procedure was as described in Example 1, with the difference that 20% isoeicosane was added to the degassed HDI. Because isoeicosane has a volatility comparable to that of HDI, working up was carried out as described in Example 1.

TABLE 2

| | Bu₄P⁺[HF₂]⁻ | | Amount | Resin | Imino- | | |
|---|---|---|---|---|---|---|---|
| | solution^{(a)} | Delta- | of resin | NCO | oxadia- | Iso- | Uret- |
| Ex. 2 | [mg] | n_{D}^{(b)} | [g] | [%] | zinediones^{(c)} | cyanurates^{(c)} | diones^{(c)} |
| A | 720 | 0.0080 | 209 | 23.4 | 54.6% | 41.7% | 3.2% |
| B | 670 | 0.0078 | 220 | 23.2 | 56.6% | 38.9% | 3.6% |
| C | 692 | 0.0077 | 228 | 23.3 | 53.7% | 39.6% | 3.1% |
| D | 692 | 0.0162 | 377 | 22.0 | 53.6% | 43.2% | 3.1% |
| E | 714 | 0.0173 | 391 | 21.7 | 51.3% | 44.0% | 3.6% |
| F | 714 | 0.0218 | 513 | 21.3 | 50.5% | 46.3% | 3.1% |
| G | 893 | 0.0217 | 498 | 21.0 | 49.2% | 47.3% | 3.1% |
| H | 982 | 0.0258 | 621 | 20.3 | 47.7% | 49.5% | 2.6% |
| I | 1004 | 0.0264 | 611 | 20.2 | 45.4% | 51.0% | 2.9% |
| J | 1071 | 0.0296 | 657 | 19.5 | 45.5% | 52.3% | 2.0% |
| K | 1071 | 0.0286 | 658 | 20.0 | 45.1% | 51.9% | 2.4% |
| L | 1116 | 0.0290 | 696 | 18.9 | 42.6% | 54.3% | 2.1% |
| M | 1205 | 0.0299 | 694 | 18.4 | 42.9% | 54.0% | 2.2% |
| N | 1272 | 0.0325 | 732 | 17.9 | 40.1% | 54.5% | 2.2% |
| O | 1272 | 0.0386 | 902 | 15.9 | 31.5% | 60.4% | 1.3% |

^{(a)}70% in iPrOH;
^{(b)}Increase in the refractive index as compared with the starting value before the first catalyst addition, the measurement was here carried out at 60° C. because the reaction mixtures became heterogeneous at 20° C. beyond a certain conversion and a determination of the n_{D}²⁰ either was not possible or gave misleading results,
^{(d)} mol-% acc. to NMR, based on the sum of the NCO secondary products formed in the modification reaction, difference to 100%: urethanes/allophanates.

### Example 3

### According to the Invention

Additive: n-Hexane (relative permittivity at 25° C./50 Hz: 1.9)

The procedure was as described in Example 1, with the difference that 20% n-hexane was added to the degassed HDI and that the n-hexane, after the respective reaction and before the vacuum distillation, was separated off at normal pressure by passage through the distillation apparatus heated to 120° C. (PE) and 140° C. (SPE) and metered into the next batch. The recyclate monomer and the polyisocyanate resin were then separated by vacuum distillation as described in Example 1.

TABLE 3

| | Bu₄P⁺[HF₂]⁻ | | Amount | Resin | Imino- | | |
|---|---|---|---|---|---|---|---|
| | solution^{(a)} | Delta- | of resin | NCO | oxadia- | Iso- | Uret- |
| Ex. 3 | [mg] | n_{D}^{(b)} | [g] | [%] | zinediones^{(c)} | cyanurates^{(c)} | diones^{(c)} |
| A | 660 | 0.0075 | 205 | 23.7 | 57.2% | 39.5% | 3.2% |
| B | 650 | 0.0072 | 198 | 23.4 | 58.4% | 38.5% | 2.8% |
| C | 682 | 0.0158 | 390 | 21.8 | 50.8% | 43.8% | 4.2% |
| D | 698 | 0.0210 | 502 | 20.8 | 49.3% | 47.5% | 2.9% |
| E | 915 | 0.0260 | 600 | 19.8 | 46.5% | 48.9% | 3.0% |
| F | 950 | 0.0285 | 668 | 19.8 | 44.2% | 52.3% | 2.9% |
| G | 1180 | 0.0315 | 730 | 18.1 | 39.5% | 54.9% | 2.4% |
| H | 1302 | 0.0392 | 915 | 15.4 | 33.3% | 62.3% | 1.8% |

^{(a)}70% in iPrOH;
^{(b)}Increase in the refractive index as compared with the starting value before the first catalyst addition, the measurement was here carried out at 60° C. because the reaction mixtures became heterogeneous at 20° C. beyond a certain conversion and a determination of the n_{D}²⁰ either was not possible or gave misleading results,
^{(d)} mol-% acc. to NMR, based on the sum of the NCO secondary products formed in the modification reaction, difference to 100%: urethanes/allophanates.

When the data given in Tables 2 and 3 are compared with those from Table 1, it is apparent that, with a comparable monomer conversion, a significant increase in the iminooxadiazinedione content in the polyisocyanate resins is achieved by the use of the non-polar additives isoeicosane or also n-hexane.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification. In this manner, Applicant(s) reserve the right to amend the claims during prosecution to add features as variously described in this specification, and such amendments comply with the requirements of 35 U.S.C. §112(a), and 35 U.S.C. §132(a).

Various aspects of the subject matter described herein are set out in the following numbered clauses:

1. Method for producing polyisocyanates comprising iminooxadiazinedione groups, wherein at least one monomeric di- and/or tri-isocyanate is oligomerised in the presence of (a) at least one catalyst, (b) at least one additive (A) having a relative permittivity at 18° C. to 30° C. of less than 4.0, (c) optionally further additives other than A.

2. Method according to clause 1, characterised in that the additive (A) is added to the monomeric di- and/or tri-isocyanate before it is brought into contact with the catalyst.

3. Method according to clauses 1 or 2, characterised in that there is used as the additive (A) a linear or branched, aliphatic, cycloaliphatic and/or araliphatic C₄-C₃₀-alkane, in particular C₅-C₃₀-alkane, or mixtures thereof, wherein the additive (A) is preferably selected from the group comprising linear, branched or cyclic butane, pentane, hexane, heptane, octane, nonane, decane, undecane, dodecane, tridecane, tetradecane, pentadecane, hexadecane, heptadecane, octadecane, nonadecane, eicosane, heneicosane, docosane, tricosane, tetracosane, pentacosane, hexacosane, heptacosane, octacosane, nonacosane, tricontane, and mixtures thereof.

4. Method according to any one of the preceding clauses, characterised in that there are used from 1 to 50 wt. %, preferably from 2 to 30 wt. %, particularly preferably from 2 to 20 wt. %, of additive (A), based on the mass of the monomeric di- and/or tri-isocyanate.

5. Method according to any one of the preceding clauses, characterised in that the additive (A) has a relative permittivity at 18° C. to 30° C. of not more than 3.5, preferably of not more than 3.0, particularly preferably of not more than 2.8 or even not more than 2.5.

6. Method according to any one of the preceding clauses, characterised in that there is used as the monomeric di- and/or tri-isocyanate an aliphatic diisocyanate, in particular hexamethylene diisocyanate (HDI), 2-methylpentane 1,5-diisocyanate, 2,4,4-trimethyl-1,6-hexane diisocyanate, 2,2,4-trimethyl-1,6-hexane diisocyanate and/or 4-isocyanatomethyl-1,8-octane diisocyanate, preferably HDI.

7. Method according to any one of the preceding clauses, characterised in that there is used as the catalyst a tetraorganyl-ammonium salt and/or phosphonium salt, wherein the anions of the tetraorganyl-ammonium salt and/or phosphonium salt are selected in particular from the group: RfCR₁R₂COO⁻, wherein Rf represents a straight-chained or branched perfluoroalkyl radical and R₁ and R₂ independently of one another represent H, straight-chained or branched organyl radicals, fluoride (F⁻), di- and/or poly-(hydrogen) fluorides ([F⁻×HF)ₘ]), wherein m has a numerical value of from 0.001 to 20, preferably from 0.1 to 20, particularly preferably from 0.5 to 20, most particularly preferably from 0.5 to 5.

8. Method according to clause 7, characterised in that the di- and/or poly-(hydrogen) fluoride ([F⁻×HF)ₘ]) is a quaternary ammonium fluoride, ammonium difluoride, ammonium trifluoride, a higher ammonium polyfluoride, a phosphonium fluoride, a phosphonium difluoride, a phosphonium trifluoride and/or a higher phosphonium polyfluoride, preferably those which can be prepared by mixing quaternary ammonium and phosphonium fluorides or hydroxides with corresponding amounts of hydrogen fluoride, optionally pre-dissolved in alcohols or water.

9. Method according to any one of the preceding clauses, characterised in that the catalyst/catalyst mixture is used in an amount of from 1 mol-ppm to 1 mol-%, preferably from 5 mol-ppm to 0.1 mol-%, in each case based on the amount of monomeric di- and/or tri-isocyanate.

10. Method according to any one of the preceding clauses, characterised in that the method is carried out in the temperature range of from 0° C. to +250° C., preferably from 20 to 180° C., particularly preferably from 40 to 150° C.

11. Method according to any one of the preceding clauses, characterised in that the oligomerisation is terminated when from 5 to 80 wt. %, preferably from 10 to 60 wt. %, of the monomeric di- and/or tri-isocyanate used has been converted.

12. Method according to clause 11, characterised in that the oligomerisation is terminated by deactivation of the catalyst, in particular by addition of an acid or of an acid derivative such as benzoyl chloride, an acid ester of acids containing phosphorus or sulfur, those acids themselves, adsorptive binding of the catalyst and subsequent separation by filtration, or combinations thereof.

13. Method according to clauses 11 or 12, characterised in that unconverted monomer is separated from the reaction mixture.

14. Reaction system for producing polyisocyanates comprising iminooxadiazinedione groups, which reaction system comprises at least one monomeric di- and/or tri-isocyanate as well as (a) at least one catalyst, (b) at least one additive (A) having a relative permittivity at 18° C. to 30° C. of less than 4.0, (c) optionally further additives other than A.

15. Use of compounds having a relative permittivity at 18° C. to 30° C. of less than 4.0 as an additive (A) for producing polyisocyanates comprising iminooxadiazinedione groups by catalyzed modification of monomeric di- and/or tri-isocyanates.

1. A method for producing polyisocyanates containing iminooxadiazinedione groups, comprising oligomerizing at least one monomeric di- and/or tri-isocyanate in the presence of
a) at least one catalyst,
b) at least one additive (A) having a relative permittivity at 18° C. to 30° C. of less than 4.0,
c) optionally further additives other than A.

2. A method according to claim 1, wherein additive (A) is added to the monomeric di- and/or tri-isocyanate before it is brought into contact with the catalyst.

3. The method according to claim 1,
wherein additive (A) is selected from the group consisting of a linear or branched, aliphatic, cycloaliphatic and/or araliphatic C₄-C₃₀-alkane.

4. The method according claim 1, wherein from 1 to 50 wt. %, of additive (A) is included, based on the mass of the monomeric di- and/or tri-isocyanate.

5. The method according to claim 1, wherein additive (A) has a relative permittivity at 18° C. to 30° C. of not more than 3.5, preferably of not more than 3.0, particularly preferably of not more than 2.8 or even not more than 2.5.

6. The method according to claim 1, wherein the monomeric di- and/or tri-isocyanate comprises an aliphatic diisocyanate.

7. The method according to claim 1, wherein as the catalyst is selected from the group consisting of a tetraorganyl-ammonium salt and/or -phosphonium salt, wherein the anions of the tetraorganyl-ammonium salt and/or -phosphonium salt are selected in particular from the group: RfCR₁R₂COO⁻, wherein Rf represents a straight-chained or branched perfluoroalkyl radical and R₁ and R₂ independently of one another represent H, straight-chained or branched organyl radicals, fluoride (F⁻), di- and/or poly-(hydrogen) fluorides ([F⁻×HF)ₘ]), wherein m has a numerical value of from 0.001 to 20.

8. The method according to claim 7, wherein that the di- and/or poly-(hydrogen) fluoride ([F⁻×HF)ₘ]) is selected from the group consisting of a quaternary ammonium fluoride, ammonium difluoride, ammonium trifluoride, a higher ammonium polyfluoride, a phosphonium fluoride, a phosphonium difluoride, a phosphonium trifluoride and/or a higher phosphonium polyfluoride.

9. The method according to claim 1, wherein that the catalyst/catalyst mixture is included in an amount of from 1 mol-ppm to 1 mol-%, based on the amount of monomeric di- and/or tri-isocyanate.

10. The method according claim 1, wherein the method is carried out in the temperature range of from 0° C. to +250° C.

11. The method according to claim 1, wherein the oligomerization is terminated when from 5 to 80 wt. %, of the monomeric di- and/or tri-isocyanate has been converted.

12. The method according to claim 11, wherein the oligomerization is terminated by addition of an acid or of an acid derivative, by addition of an acid ester of acids containing phosphorus or sulfur, those acids themselves, or by adsorptive binding of the catalyst and subsequent separation by filtration, or combinations thereof.

13. The method according to one of claims 11 and 12, wherein unconverted monomer is separated from the reaction mixture.

14. A reaction system for producing polyisocyanates comprising iminooxadiazinedione groups, the reaction system comprising at least one monomeric di- and/or tri-isocyanate,
a) at least one catalyst,
b) at least one additive (A) having a relative permittivity at 18° C. to 30° C. of less than 4.0,
c) optionally further additives other than A.

15. The method according to claim 3, wherein additive (A) is selected from the group consisting of linear, branched or cyclic butane, pentane, hexane, heptane, octane, nonane, decane, undecane, dodecane, tridecane, tetradecane, pentadecane, hexadecane, heptadecane, octadecane, nonadecane, eicosane, heneicosane, docosane, tricosane, tetracosane, pentacosane, hexacosane, heptacosane, octacosane, nonacosane, tricontane, and mixtures thereof.

16. The method according claim 4, wherein from 2 to 30 wt. %, of additive (A) is included, based on the mass of the monomeric di- and/or tri-isocyanate.

17. The method according claim 4, wherein from 2 to 20 wt. %, of additive (A) is included, based on the mass of the monomeric di- and/or tri-isocyanate.

18. The method according to claim 5, wherein additive (A) has a relative permittivity at 18° C. to 30° C. of not more than 3.0

19. The method according to claim 5, wherein additive (A) has a relative permittivity at 18° C. to 30° C. of not more than 2.8.

20. The method according to claim 5, wherein additive (A) has a relative permittivity at 18° C. to 30° C. not more than 2.5.

21. The method according to claim 6, wherein the monomeric di- and/or tri-isocyanate is selected from the group consisting of hexamethylene diisocyanate (HDI), 2-methylpentane 1,5-diisocyanate, 2,4,4-trimethyl-1,6-hexane diisocyanate, 2,2,4-trimethyl-1,6-hexane diisocyanate and 4-isocyanatomethyl-1,8-octane diisocyanate.

22. The method according to claim 6, wherein the monomeric di- and/or tri-isocyanate comprises hexamethylene diisocyanate (HDI).
